(19) United States
(12) Patent Application Publication
(10) Pub. No.: US 2025/0241734 A1
(43) Pub. Date: Jul. 31, 2025

(54) SYSTEM AND METHOD FOR GLAZING DENTAL RESTORATIONS WITH CERAMIC INK VIA INKJET PRINTER

(71) Applicant: Zima International, Inc. d/b/a Dandy, New York, NY (US)

(72) Inventors: Patrick Moore, New York, NY (US); Alec Tieferthal, New York, NY (US); Kenn Butler, New York, NY (US); Kevin myers, New York, NY (US); Daniel Hanover, NBew York, NY (US); Oluwatoniloba Oloko, New York, NY (US); Srinivasan Sundararajan, New York, NY (US)

(21) Appl. No.: 19/041,386

(22) Filed: Jan. 30, 2025

Related U.S. Application Data
(60) Provisional application No. 63/627,670, filed on Jan. 31, 2024.

(51) Int. Cl. A61C 13/08; B41J 3/407; B41J 29/00; B41M 5/00

(52) U.S. Cl. CPC A61C 13/082; B41J 3/4073; B41J 29/00; B41M 5/0088

(57) ABSTRACT

Disclosed are systems and methods of a decision intelligence (DI)-based computerized framework that automatically and/or dynamically implements a color algorithm for performing staining and glazing dental restorations. The framework operates by leveraging determined color variations through AI/ML-based analysis that generate detailed color maps, which can be translated into precise printing instructions controlling the mixture and layering of dental-grade glazing materials. A printing system can employ multiple print heads with varying base colors and opacity levels to create microscopic color gradients and translucency effects matching natural tooth enamel. The disclosed automated processing applies personalized color patterns in microscopically thin layers, ensuring consistent glaze thickness while replicating regional variations across the tooth surface. Such approach eliminates manual glazing variability while achieving superior customization compared to traditional pre-set patterns, resulting in dental restorations that integrate seamlessly with existing teeth.

### CROSS-REFERENCE TO RELATED APPLICATIONS

The instant application claims the benefit of priority from U.S. Provisional Patent Application No. 63/627,670, filed Jan. 31, 2024, which is incorporated herein by reference in its entirety.

### FIELD OF THE DISCLOSURE

The present disclosure is generally related to dental restoration processing, and more particularly, to a decision intelligence (DI)-based computerized framework that automatically and/or dynamically controls and executes a color algorithm for performing staining and glazing of dental restorations.

### SUMMARY OF THE DISCLOSURE

By way of background, glazing of a dental restoration is an important finishing step that improves the dental restoration's resistance to damage, and that makes the dental restoration appear more realistic. Generally, this glazing is performed manually according to one of several pre-set coloration patterns.

Such manual glazing process for dental restorations faces several significant technical and practical limitations that impact both production and outcomes. The inherent variability of manual application creates consistency challenges between technicians and even between restorations by the same technician, making standardization difficult and affecting quality control. The time-intensive nature of the process, requiring skilled technicians to carefully apply and blend colors according to predetermined patterns, increases production costs and turnaround times. The heavy reliance on technician expertise and artistic ability creates bottlenecks and makes training new technicians lengthy, while manual coloration patterns may not adequately capture natural tooth variation.

Manual application also makes it challenging to maintain consistent glaze thickness and coverage, affecting both aesthetics and protective properties. These technical limitations manifest in real-world impacts across business operations, clinical outcomes, and patient experience. Dental laboratories face higher production costs, extended timelines, and increased training requirements, while clinical outcomes can vary significantly between providers. Patients may experience longer wait times, higher costs, and potential dissatisfaction with aesthetic results or restoration longevity.

Indeed, the industry as a whole faces barriers to scaling operations, increased reliance on skilled technicians, limited standardization, and higher overhead costs affecting market competitiveness. These challenges point to opportunities for technological advancement in the glazing process to improve consistency, efficiency, and quality.

To that end, the disclosed systems and methods provide a computerized framework for utilizing an inkjet printer directed by proprietary color algorithm and/or design software that operates to stain and glaze a ceramic dental restoration with ceramic ink to, for example, as discussed in more detail below, mimic the coloration and translucency of an original or real tooth.

According to some embodiments, as discussed herein, the disclosed framework functions via integration with and/or implementation with three-dimensional (3D) printing technology to create personalized and/or dynamically curated glazing color matches for each patient. As discussed in more detail below, according to some embodiments, the disclosed framework operates by capturing detailed digital images of the patient's existing teeth under controlled lighting conditions, analyzing subtle variations in color, transparency, fluorescence, surface characteristics, and the like, or some combination thereof. Such images can be processed through sophisticated computer vision algorithms, for example, that break down the tooth's color composition into precise color maps, identifying the intricate patterns of hue, saturation, and value that make each person's teeth unique. The proprietary algorithm then translates this detailed color analysis into specific printing instructions, controlling the precise mixture and layering of dental-grade glazing materials to replicate these natural variations. The printing system utilizes multiple print heads with different base colors and opacity levels, allowing for microscopic color gradients and translucency effects that mirror natural tooth enamel.

According to some embodiments, the disclosed framework can function by accounting for factors that include, but are not limited to, material thickness, light diffusion properties, the interaction between different color layers, and the like, to ensure the final printed result accurately matches the patient's natural teeth. By incorporating artificial intelligence and/or machine learning (AI/ML) capabilities, the framework can continuously improve its color matching accuracy by learning from previous cases and adjusting for variations in printing conditions.

Accordingly, in some embodiments, the printing process applies such personalized color patterns in microscopically thin layers, building up the glaze coating with precise control over thickness and coverage. Such automated approach eliminates the variability of manual glazing while achieving a higher level of customization than traditional pre-set patterns could offer. The framework can also account for regional variations across the tooth surface, replicating the subtle color transitions found in natural teeth, from the slightly darker cervical area to the more translucent incisal edge. Thus, implementation of the disclosed systems and methods can result in a dental restoration that seamlessly integrates with the patient's existing teeth, providing both aesthetic excellence and consistent protective properties.

According to some embodiments, a method is disclosed that automatically and/or dynamically controls and executes a color algorithm for performing glazing dental restorations. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method that automatically and/or dynamically controls and executes a color algorithm for performing glazing dental restorations.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a perspective view of a system for applying ceramic glaze to a dental restoration using an inkjet printer head according to some embodiments of the present disclosure;

FIG. 2A is a side view of the system of FIG. 1 in a first position according to some embodiments of the present disclosure;

FIGS. 2B and 2C provide non-limiting example views of the system of FIG. 1 according to some embodiments of the present disclosure;

FIG. 2D provides a non-limiting example view of the system of FIG. 1 according to some embodiments of the present disclosure;

FIG. 3 is a side view of the system of FIG. 1 in a second position according to some embodiments of the present disclosure;

FIG. 4A is a perspective view of the dental restoration and the inkjet printer head of FIG. 1 in a first orientation according to some embodiments of the present disclosure;

FIG. 4B is a side view of the dental restoration and the inkjet printer head of FIG. 1 in a second orientation according to some embodiments of the present disclosure;

FIG. 4C is a side view of the dental restoration and the inkjet printer head of FIG. 1 in a third orientation according to some embodiments of the present disclosure;

FIG. 4D is a side view of the dental restoration and the inkjet printer head of FIG. 1 in a fourth orientation according to some embodiments of the present disclosure;

FIG. 5 depicts example views of a restoration component under a scanner according to some embodiments of the present disclosure;

FIG. 6 is a block diagram of an example configuration within which the systems and methods disclosed herein could be implemented according to some embodiments of the present disclosure;

FIG. 7 is a block diagram illustrating components of an exemplary system according to some embodiments of the present disclosure; and

FIG. 8 illustrates an exemplary workflow according to some embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. By way of background, dental restorations may be initially manufactured (or machined) as a monolithic component having a single color. For example, when the dental restoration is formed by systematically-machining away portions of a ceramic block, the resultant dental restoration may be monochromatic and can have the color of the original ceramic block. Similarly, when the dental restoration is additively-manufactured through the layering of 3D-printed material, the manufactured dental restoration is simply the color of the material used to print the restoration. Given that cosmetic appearance of the dental restoration is an important component, the monochromatic nature of the unglazed dental restoration causes issues by being clearly distinguishable from the surrounding real teeth that are generally multi-chromatic due to years (or decades) of use and staining.

To address the conventional cosmetic failings in the art (as well as to increase the strength of the dental restoration), a layer of glaze is applied to dental restorations after manufacture (or machining) and before insertion into a patient's mouth. Conventionally, a manual worker or artisan is tasked with applying this layer of glaze, due to the level of detail required for precisely mimicking the color of real teeth, as well as mechanical limitations imposed by the time of glaze used. However, the limited amount of time and limited resources of each worker often requires that the coloration pattern be chosen from one of a limited number of options. As such, not only is this manual-glazing process time-consuming, accuracy of such conventional approach is also limited by the skill or capacity of the glaze-applier.

Accordingly, there exists a need for an automated system that is capable of determining an appropriate coloration pattern and applying the appropriate pattern using an inkjet head that dispenses ceramic ink (e.g., a mixture of ceramic stain and glaze). By automating the process, the limitations previously imposed by human labor—timeliness and inaccuracy—are reduced if not outright eliminated. Not only will this increase the production capability for dental restorations, but this system will also improve the aesthetic quality of each restoration produced.

Although reference is made herein to these techniques being applied to restorations (e.g., crowns, bridges, implants, etc.), this disclosure should not be read as limited to only restorations, and should be read to include any relevant or known or to be known dental appliance, such as removable appliances (e.g., partial dentures, full dentures, etc.).

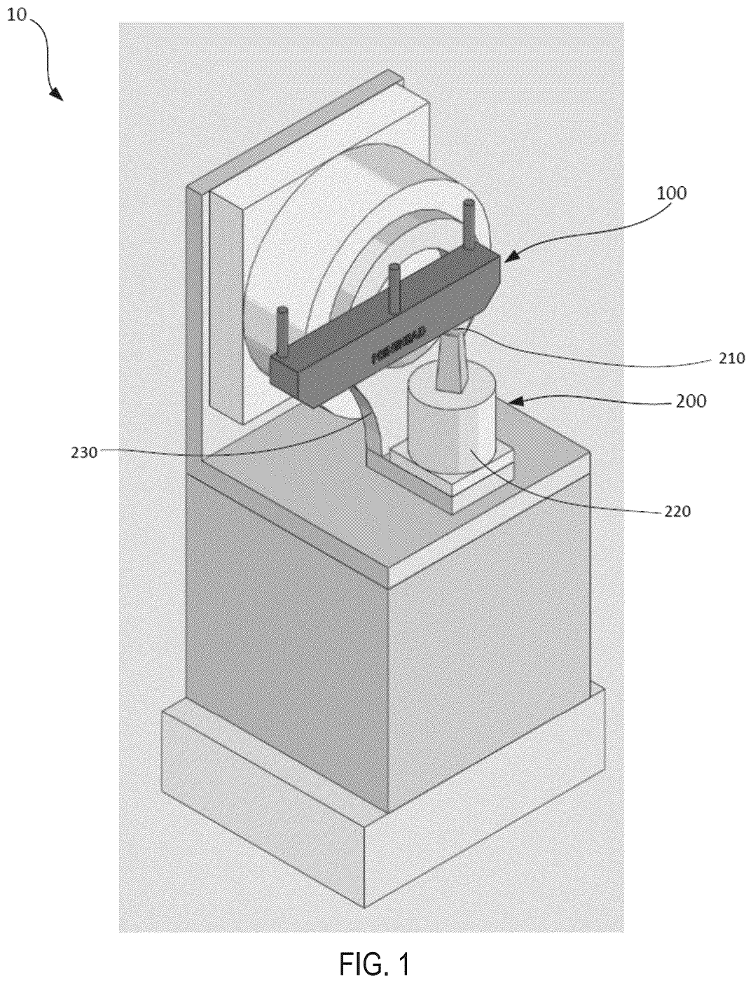

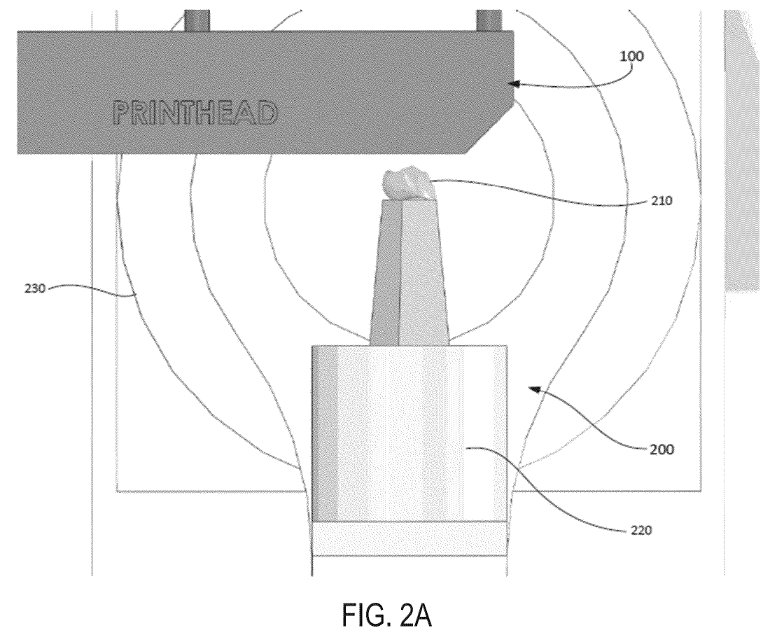

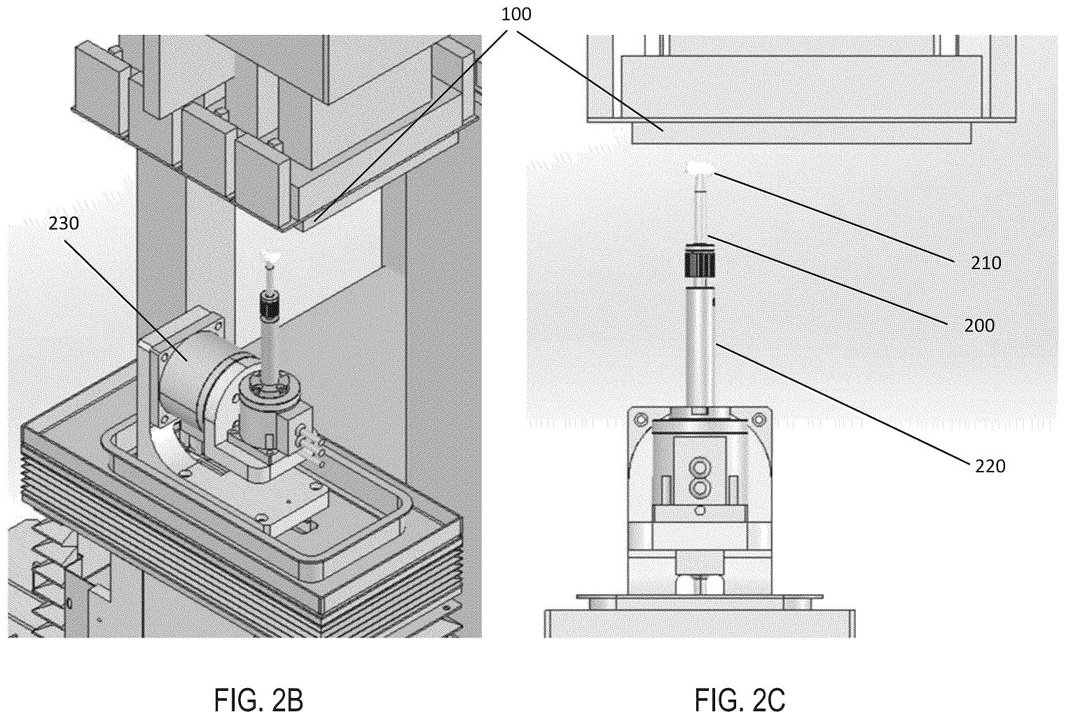

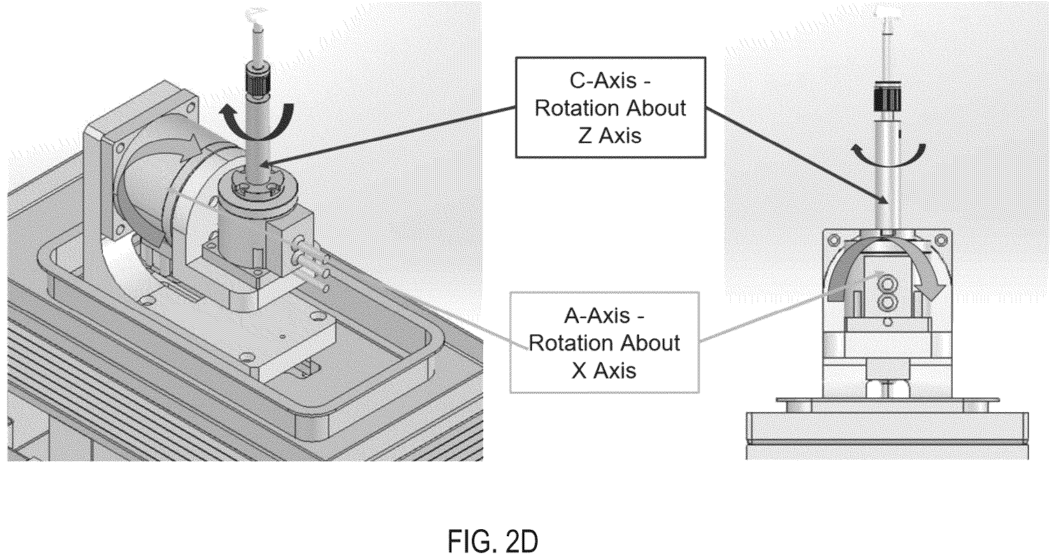

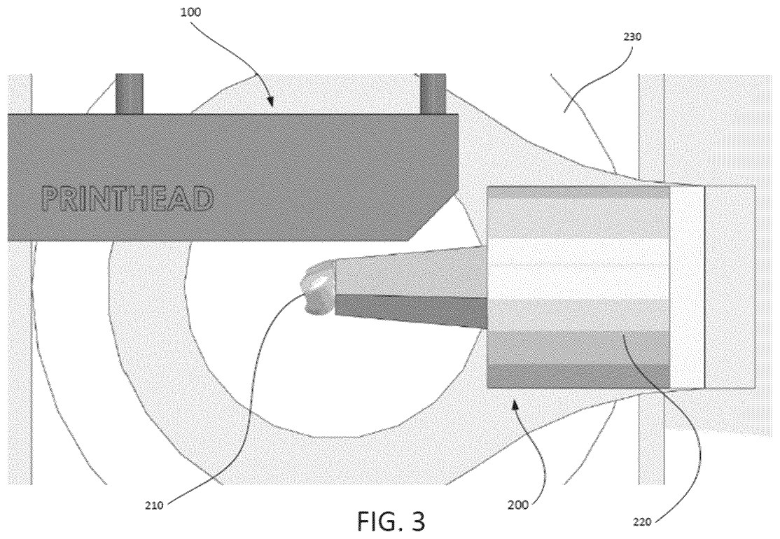

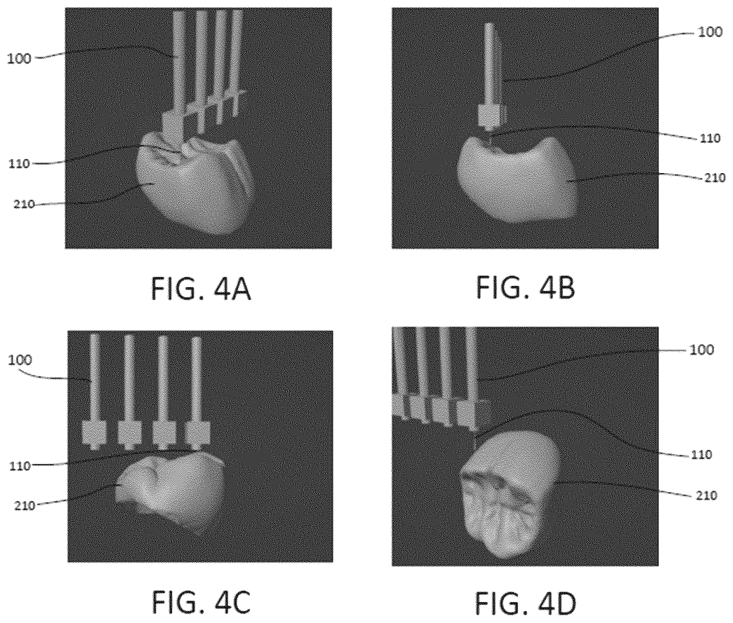

As shown in FIG. 1, a system 10 for applying a ceramic glaze to a dental restoration 210 using an inkjet printer is shown to include an inkjet printer head 100 and a crown holder 200. The crown holder 200 is shown to include a restoration 210, a base 220, and a rotation mechanism 230. In some embodiments (including the embodiment shown), the inkjet printer head 100 has a fixed position, such that the location of the glaze application (e.g., droplet stream 110) from the inkjet printer head 100 has a fixed origin. In these embodiments, the different sides of the restoration 210 are exposed to the droplet stream via movement of the restoration 210 itself, which may be accomplished via movement of the base 220 about a Y-axis (relative to the views of FIGS. 2A-2D and 3) and/or the rotation mechanism 230 about a Z-axis (relative to the views of FIGS. 2A-2D and 3). FIGS. 4A-D illustrate various positions of the restoration 210 relative to the inkjet printer head 100. In other embodiments, the inkjet printer head 100 may be capable of movement while the restoration 210 is stationary, such that the inkjet printer head 100 moves about the restoration 210 in order to apply glaze to each portion of the restoration 210, as needed. In further embodiments, both the inkjet printer head 100 and the restoration 210 are movable, such that each portion of the restoration 210 is glazed through a combination of both components moving.

According to some embodiments, the movement capability of the crown holder 200 is illustrated in FIGS. 2A-2D and 3. FIG. 2A shows the crown holder 200 in a first position in which the base 220 is positioned at 6 o'clock relative to a “clock” defined by the rotation mechanism 230. FIG. 3 shows the crown holder 200 in a second position in which the base 220 is positioned at 3 o'clock relative to the “clock” defined by the rotation mechanism 230. The transition from the first position of FIG. 2A to the second position of FIG. 3 is accomplished via rotation of the rotation mechanism 230. Because the rotation mechanism 230 is shaped such that the restoration 210 is substantially in the center of the rotation mechanism 230, a distance between the inkjet printer head 100 and the restoration 210 remains substantially the same throughout rotation of the rotation mechanism 230. Similarly, the restoration 210 is positioned in the relative center of the base 220, so the rotation of the base 220 does not affect the distance between the restoration 210 and the inkjet printer head 100. Maintaining a consistent distance between the restoration 210 and the inkjet printer head 100 is important to the functionality of the system 10, as inconsistencies can cascade into inconsistent coloration.

As depicted in FIGS. 2B and 2C, depicted are additional views of non-limiting example embodiments of the depictions in FIG. 2A. FIG. 2B depicts a view of the printhead 100 and rotational mechanism 230; and FIG. 2C depicts a view of the first position as provided in FIG. 2A, discussed supra.

In FIGS. 2E, provided is a non-limiting embodiment of functionality of the rotational mechanism 230, as discussed above respective to FIG. 2A and FIG. 3, and in more detail below.

Accordingly, such disclosure in FIGS. 2A-2D and FIG. 3 provide non-limiting printing methodologies for which dynamic movement of a restoration under a printer head 100 (or heads) can be utilized/implemented.

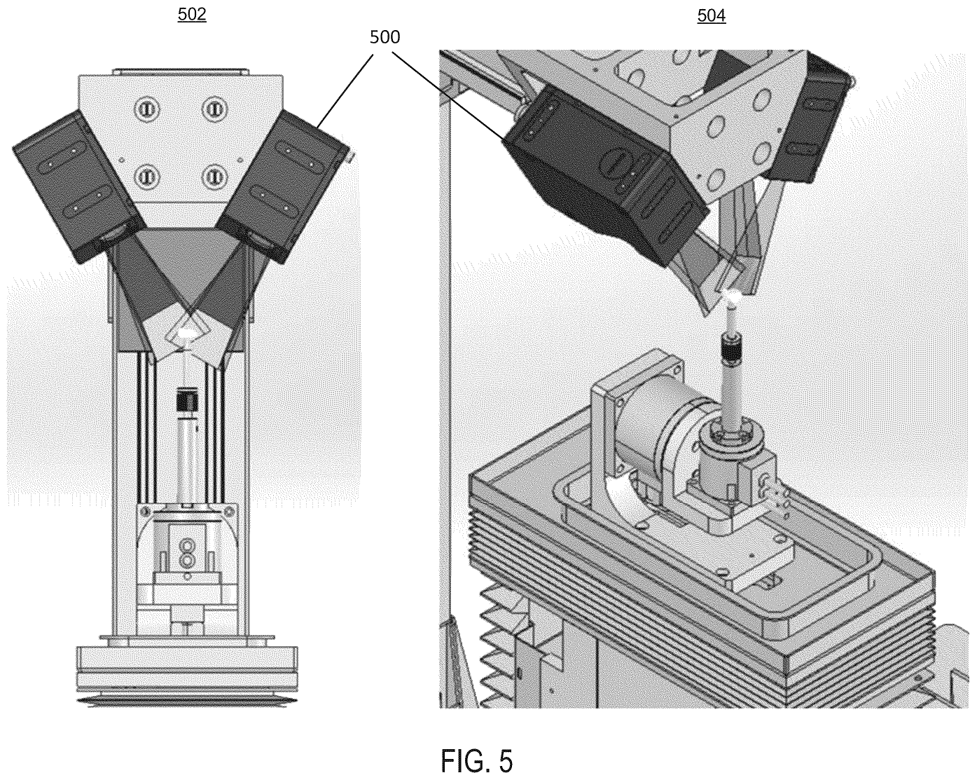

Turning to FIG. 5, depicted is a non-limiting example embodiment of restoration 210 being scanned by a scanner 500. Example view 502 and view 504 provide example depicts of a scanner being utilized to capture information related to a dental restoration component. That is, according to some embodiments, in order to determine the coloration pattern to be printed, the system 10 may utilize a scanner 500 (e.g., an optical scanner) to generate a scan of a real tooth (or teeth) of the patient. The scan may be a 3D rendering of the scanned tooth (or teeth) and may be analyzed in order to derive a coloration pattern for printing. In some embodiments, the scan may be taken of the teeth that are positioned on either side of the position for the dental restoration (e.g., the gap into which the dental restoration will be fixed), such that the coloration pattern is derived based on those teeth closest to the dental restoration in order to maximize the aesthetic consistency of the dental restoration.

The derived coloration pattern may be a two-dimensional (2D) map or plot that defines a number of points and a color profile (e.g., tone, hue, base color, etc.) that corresponds to each point. The 2D map may then be virtually applied to a 3D rendering of the dental restoration (e.g., like a vinyl wrap for a vehicle), with adjustments made—either automatically or via human input—in order to determine a coloration profile for the entire dental restoration. In some embodiments—such as those in which the dental restoration is a near-exact replica of another tooth (e.g., the dental restoration for a right incisor is based on the left incisor), the coloration profile may be derived directly from the other tooth.

Once the 3D coloration profile is established, the system 10 may determine a rotation profile for the crown holder 200 and a print pattern for the inkjet printer head 100. The rotation profile may include commands for moving the base 220 and the rotation mechanism 230 that, in coordination with the print pattern, ensure that each portion of the dental restoration 210 is exposed to ceramic ink. The print pattern may include commands for dispensing ceramic ink via the inkjet printer head 100 at commanded quantities, qualities (e.g., color combinations), and timings of ink. In particular, the print pattern may account for changes in topography across the surface of the dental crown by affecting the speed and/or volume of a particular droplet stream. Both the rotation profile and the print pattern may be optimized in order to reduce the overall time spent applying glaze.

This system overcomes several notable technical issues. First, the size (about 10 microns) of the droplets of the manually-applied ceramic ink exceeds the capability (<1 micron) of most inkjet printing heads. In some embodiments, the system makes use of a specially-designed inkjet head that enables passage for the larger droplets. In some embodiments, a different formulation for the ceramic ink is developed that performs a similar function to the original ceramic ink but at a reduced size capable of use with standard inkjet heads.

Second, the cyan-yellow-magenta-white (CYMW) color palette that is standard for inkjet printing may not satisfactorily match the various shades of multichromatic real teeth. In some embodiments, the system utilizes a bespoke collection of ink colors that, either alone or in combination, is capable of matching any required coloration pattern.

Third, the applied ceramic glaze must also match the translucency (e.g., reflectivity) of a real tooth, in addition to the coloration pattern. To this end, the system may apply various amounts of clear and/or blue (which appears translucent as ceramic) ink in addition to the quantities of ink applied for the specific coloration pattern. Because these translucent layers are different from the color layers, the system accounts for both when determining the printing pattern, as a too-thick layer of glaze can impact the fit, appearance, or performance of the dental restoration.

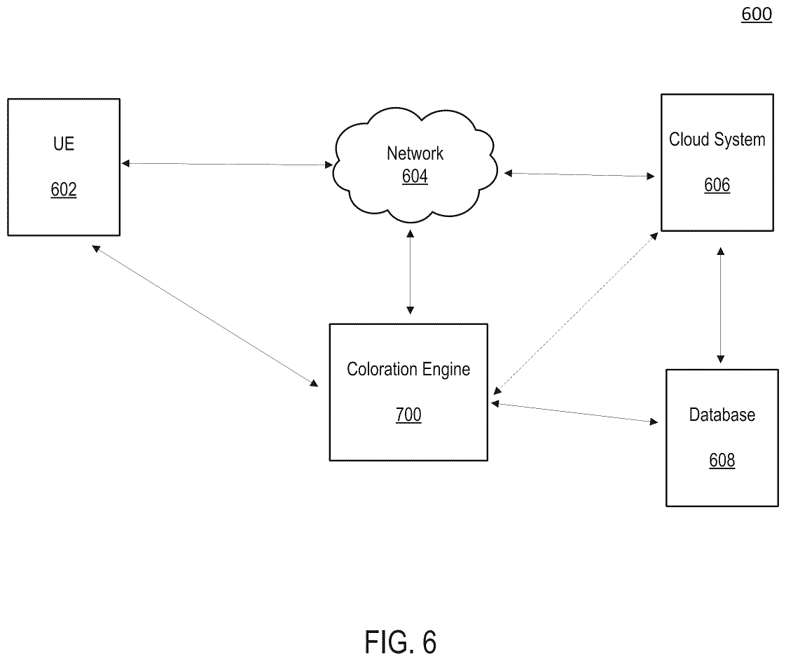

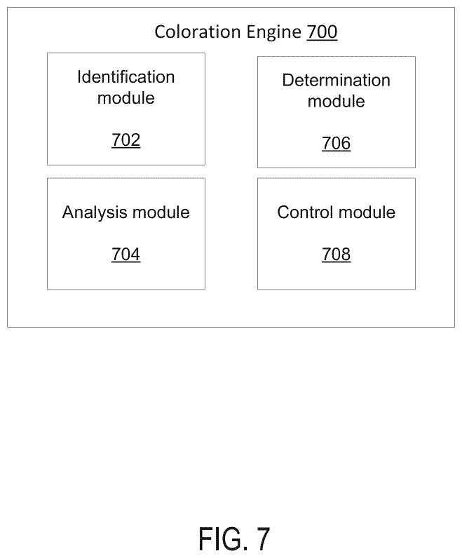

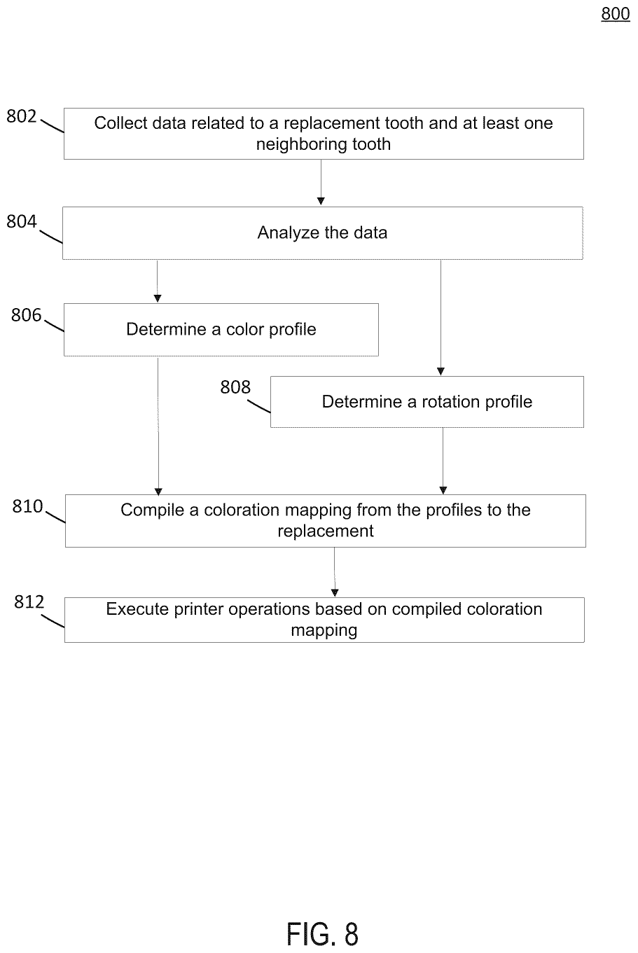

Turning to FIGS. 6-8, disclosed are non-limiting embodiments for implementing the above functional components of the disclosed system 10 as provided via FIGS. 1-5. Accordingly, FIG. 6 provides an example operating environment for which such system(s) can function; FIG. 7 provides engine 700 which represents the computerized framework (e.g., color algorithm) for providing the novel color matching and glazing, as discussed herein; and FIG. 8 provides Process 800 which details non-limiting example embodiments for execution of engine 700 within a runtime environment.

With reference to FIG. 6, a system 600, similar to system 10, discussed supra, is depicted which includes user equipment (UE) 602, network 604, cloud system 606, database 608 and coloration engine 700. It should be understood that while system 600 is depicted as including such components, it should not be construed as limiting, as one of ordinary skill in the art would readily understand that varying numbers of UEs, cloud systems, databases and networks, inter alia, can be utilized; however, for purposes of explanation, system 600 is discussed in relation to the example depiction in FIG. 6.

According to some embodiments, UE 602 can represent the printer system (e.g., system 10 and/or the printer from FIGS. 1-5, discussed supra). In some embodiments, UE 602 can correspond to any other device that can connect to system 10 and control its operation, such as, for example, a mobile phone, tablet, laptop, sensor, Internet of Things (IoT) device, autonomous machine, and any other device equipped with a cellular or wireless or wired transceiver.

In some embodiments, a peripheral device (not shown) can be connected to UE 602, and can be any type of peripheral device, such as, but not limited to, a wearable device (e.g., smart watch), printer, display, speaker, sensor, and the like. In some embodiments, a peripheral device can be any type of device that is connectable to UE 602 via any type of known or to be known pairing mechanism, including, but not limited to, Wi-Fi, Bluetooth™, Bluetooth Low Energy (BLE), NFC, and the like.

In some embodiments, network 604 can be any type of network, such as, but not limited to, a wireless network, cellular network, the Internet, and the like (as discussed above). Network 604 facilitates connectivity of the components of system 600, as illustrated in FIG. 6.

According to some embodiments, cloud system 606 may be any type of cloud operating platform and/or network based system upon which applications, operations, and/or other forms of network resources may be located. For example, system 606 may be a content provider, service provider and/or network provider from where services and/or applications may be accessed, sourced or executed from. For example, system 606 can represent the cloud-based architecture associated with a system provider (e.g., healthcare company, private company, and the like, for example), which has associated network resources hosted on the internet or private network (e.g., network 604), which enables (via engine 700) the dental management discussed herein.

In some embodiments, cloud system 606 may include a server(s) and/or a database of information which is accessible over network 604. In some embodiments, a database 608 of cloud system 606 may store a dataset of data and metadata associated with local and/or network information related to a user(s) of UE 602 and the UE 602, and the services and applications provided by cloud system 606 and/or coloration engine 700.

In some embodiments, for example, cloud system 606 can provide a private/proprietary management platform, whereby engine 700, discussed infra, corresponds to the novel functionality system 606 enables, hosts and provides to a network 604 and other devices/platforms operating thereon.

In some embodiments, the exemplary computer-based systems/platforms, the exemplary computer-based devices, and/or the exemplary computer-based components of the present disclosure may be specifically configured to operate in a cloud computing/architecture 606 such as, but not limiting to: infrastructure a service (IaaS), platform as a service (PaaS), and/or software as a service (SaaS) using a web browser, mobile app, thin client, terminal emulator or other endpoint.

According to some embodiments, database 608 may correspond to a data storage for a platform (e.g., a network hosted platform, such as cloud system 606, as discussed supra), a plurality of platforms, and/or UE 602. Database 608 may receive storage instructions/requests from, for example, engine 700 (and associated microservices), which may be in any type of known or to be known format, such as, for example, standard query language (SQL). According to some embodiments, database 608 may correspond to any type of known or to be known storage, for example, a memory or memory stack of a device, a distributed ledger of a distributed network (e.g., blockchain, for example), a look-up table (LUT), and/or any other type of secure data repository.

Coloration engine 700, as discussed above and further below in more detail, can include components for the disclosed functionality. According to some embodiments, coloration engine 700 may be a special purpose machine or processor, and can be hosted by a device on network 604, within cloud system 606, on UE 602. In some embodiments, engine 700 may be hosted by a server and/or set of servers associated with cloud system 606.

According to some embodiments, as discussed in more detail below, coloration engine 700 may be configured to implement and/or control a plurality of services and/or microservices, where each of the plurality of services/microservices are configured to execute a plurality of workflows associated with performing the disclosed dental management. Non-limiting embodiments of such workflows are provided below.

According to some embodiments, as discussed above, coloration engine 700 may function as an application provided by cloud system 606. In some embodiments, engine 700 may function as an application installed on a server(s), network location and/or other type of network resource associated with system 606. In some embodiments, engine 700 may function as an application installed and/or executing on UE 602. In some embodiments, such application may be a web-based application accessed by UE 602 and/or devices over network 604 from cloud system 606. In some embodiments, engine 700 may be configured and/or installed as an augmenting script, program or application (e.g., a plug-in or extension) to another application or program provided by cloud system 606 and/or executing on UE 602.

As illustrated in FIG. 7, according to some embodiments, coloration engine 700 includes identification module 702, analysis module 704, determination module 706 and control module 708. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 700 and each of its modules, and their role within embodiments of the present disclosure will be discussed below.

Turning to FIG. 8, Process 800 provides non-limiting example embodiments of the disclosed framework that provides features, capabilities and/or functionality for enhanced and/or improved dental restoration.

According to some embodiments, as discussed herein and more detail below, Process 800 provides steps for the disclosed personalized color matching and application for dental restorations through advanced color mapping, simulation, and multi-layer printing techniques. The disclosed framework processes input data from multiple sources, including direct digital imaging of patients' existing teeth and standardized shade references, to generate precise color and optical property specifications. As discussed herein, such approach fundamentally differs from traditional methods by treating color application as a complex, multi-dimensional optimization problem rather than a simple surface treatment.

According to some embodiments, the framework's architecture includes several integrated computational components. In some embodiments, the core color mapping engine analyzes input sources to create detailed spatial maps of optical properties, encoding multiple channels of information including base coloration, translucency, and surface characteristics. Such properties can be mapped onto a 3D surface representation using sophisticated texture mapping techniques, where each point can contain multiple layers of optical information. Unlike conventional printing systems, the framework employs a proprietary color space transformation algorithm that accounts for the unique properties of dental materials and their optical interactions.

According to some embodiments, as discussed herein, a key innovation of the disclosed framework's configuration and operation involves inverse rendering capabilities, which simulate the interaction between substrate materials, glazing layers, and light to predict the final optical appearance. Such simulation considers material thickness, opacity gradients, and the natural optical properties of teeth to determine precise material deposition parameters. The framework incorporates AI/ML components that continuously refine color matching accuracy by analyzing relationships between input specifications and measured outcomes, enabling adaptation to variations in base material properties and printing conditions.

According to some embodiments, the disclosed color optimization operations employed by the framework can be effectuated via sophisticated AI/ML algorithms that can translate desired optical properties into specific material combinations and layering sequences. Unlike traditional dental color matching systems that rely on premixed materials approximating standardized shades, the disclosed framework dynamically calculates precise mixtures of base materials needed to achieve target optical properties. For example, such operations can include accounting for phenomena such as metamerism, where different material combinations can produce visually identical results under specific lighting conditions, enabling more robust and naturalistic results.

In some embodiments, the framework's material deposition planning component generates detailed instructions for multi-layer printing, controlling parameters such as layer thickness, material ratios, and deposition patterns. This enables the creation of complex optical effects such as subsurface scattering and natural color gradients that mimic the internal structure of natural teeth. The framework accounts for material-specific properties such as opacity, translucency, and light diffusion characteristics, adjusting deposition strategies to achieve desired optical outcomes while maintaining structural integrity.

Moreover, in some embodiments, the framework includes calibration and validation operations which can function to maintain color accuracy by continuously monitoring and adjusting for variations in base material properties, environmental conditions, and equipment performance, among other properties and/or characteristics. The framework can employ color measurement and comparison algorithms to validate output against target specifications, automatically initiating adjustment cycles when results fall outside acceptable parameters. This, for example, ensures consistent, predictable results despite variations in input materials or conditions.

In some embodiments, the framework incorporates advanced visualization capabilities that enable real-time preview and adjustment of restoration appearances. This includes simulation of different lighting conditions and viewing angles, allowing for optimization of both aesthetic and functional properties. The visualization system accounts for complex optical phenomena such as subsurface scattering and edge effects, providing accurate predictions of final appearance under various real-world conditions.

In some embodiments, material optimization algorithms within the framework can be implemented to consider both aesthetic and functional requirements, ensuring that color and optical property specifications maintain compatibility with structural and durability requirements. This includes analysis of material thickness constraints, thermal properties, and wear characteristics, adjusting color specifications to maintain optimal performance while achieving desired aesthetic outcomes.

As discussed herein, in some embodiments, the framework's adaptability is enhanced through a comprehensive database of material optical properties, color interaction models, and historical performance data. Such knowledge base enables the framework to make intelligent decisions about material selection and application strategies, considering factors such as material compatibility, environmental stability, and long-term color stability. The database (e.g., database 608) can be continuously updated through AI/ML algorithms that analyze relationships between specified parameters and measured outcomes.

In some embodiments, quality control mechanisms can be integrated throughout the framework, monitoring each stage of the process from initial color analysis through final validation. These mechanisms employ statistical process control techniques to identify and correct deviations from target specifications, ensuring consistent, predictable results. The framework maintains detailed records of process parameters and outcomes, enabling continuous improvement of color matching and application strategies.

In some embodiments, advanced error correction algorithms can be utilized to enable the framework to compensate for various sources of variation and uncertainty. This includes adjustment for differences in material properties between batches, environmental effects on color development, and variations in substrate characteristics. The framework can automatically modify process parameters to maintain consistent results despite these variations, ensuring reliable color matching across different production conditions.

In some embodiments, the framework's output generation operations can produce comprehensive specifications for material deposition, including detailed instructions for layer composition, thickness, and sequencing. Such specifications account for both the aesthetic requirements of the restoration and the practical constraints of the production process, ensuring that desired optical properties can be achieved reliably and efficiently.

According to some embodiments, Step 802 of Process 800 can be performed by identification module 702 of coloration engine 700; Step 804 can be performed by analysis module 704; Steps 806 and 808 can be performed by determination module 706; and Steps 810 and 812 can be performed by control module 708.

According to some embodiments, Process 800 begins with Step 802 where engine 700 collects data related to the replacement tooth, a previous tooth, at least one neighboring tooth, or some combination thereof. For example, the data can relate to a tooth that was previously within the patient, for which the replacement is processed, as discussed herein, to match. In another example, such data can correspond to an adjacent tooth, or the matching tooth on the other side of the mouth (e.g., matching incisors, for example). According to some embodiments, such data is sourced from multiple input modalities, including direct digital imaging, intraoral scans, and standardized shade references. Engine 700 processes this input to extract detailed information about the optical and structural characteristics of the patient's existing teeth. This data forms the foundation for subsequent analysis, ensuring that the replacement tooth integrates seamlessly with the surrounding dentition. The collected data includes critical parameters such as base coloration, translucency gradients, surface texture, and optical phenomena like light scattering and reflection.

As discussed herein, the ability to capture detailed and multi-dimensional data is pivotal for personalization. By including information about the neighboring teeth, the framework ensures that the replacement tooth's appearance harmonizes with the patient's natural dentition. The inclusion of standardized shade references further improves the accuracy of the collected data, providing a benchmark for comparison and validation.

In Step 804, engine 700 employs advanced AI/ML algorithms to analyze the collected data. In some embodiments, engine 700 operates to process such input to create detailed spatial maps of optical properties. These maps encode multiple channels of information, including, but not limited to, base coloration, translucency gradients, surface texture characteristics, and the like. The AI/ML components continuously refine their accuracy by learning from historical data and real-world outcomes.

In some embodiments, the analysis in Step 804 can function by treating color matching as a multi-dimensional optimization operation, considering factors such as, but not limited to, the refractive indices of dental materials, light absorption spectra, scattering coefficients., and the like, or some combination thereof. Unlike traditional methods that rely on visual matching, the implemented AI/ML algorithms identify intricate patterns and relationships within the data. For example, the framework can detect subtle variations in translucency and surface texture that influence the overall appearance of the tooth. Moreover, the incorporation of AI/ML enables the framework to adapt to variations in input data, such as differences in imaging quality or material properties. This adaptability ensures that the analysis remains robust and reliable across diverse clinical scenarios. By leveraging the power of AI/ML, the framework achieves a level of precision and consistency that surpasses traditional approaches.

Accordingly, in some embodiments, such analysis of Step 804 can involve engine 700 implementing any type of known or to be known computational analysis technique, algorithm, mechanism or technology to analyze the collected information from Step 802.

In some embodiments, engine 700 may include a specific trained AI/ML model, a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, engine 700 may be configured to utilize one or more AI/ML techniques chosen from, but not limited to, computer vision, feature vector analysis, decision trees, boosting, support-vector machines, neural networks, nearest neighbor algorithms, Naive Bayes, bagging, random forests, logistic regression, physics based rendering, differentiable rendering, inverse rendering, neural rendering, and the like. By way of a non-limiting example, engine 700 can implement an XGBoost algorithm for regression and/or classification to analyze the dental and/or patient data, as discussed herein.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
e. apply the trained model to process the newly-received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

In Step 806, engine 700 determines a color profile based on the results of the data analysis. As discussed above, a color profile represents a comprehensive specification of the target optical properties, including, but not limited to, hue, saturation, brightness, translucency and the like. The framework's proprietary color space transformation algorithm plays a crucial role in this step, translating the analyzed data into a precise and actionable color profile, as discussed above.

In some embodiments, the color profile accounts for the 3D nature of dental restorations and the unique optical properties of dental materials. By incorporating parameters such as, for example, material refractive indices and light interaction characteristics, engine 700 can ensure that the color profile accurately represents the desired appearance. In some embodiments, Step 806's operation by engine 700 can consider and/or account for phenomena such as metamerism, ensuring that the restoration appears natural under various lighting conditions.

In some embodiments, the color profile determination in Step 806 can balance aesthetic and functional requirements. For example, engine 700 may adjust the color profile to account for structural considerations, such as the thickness of the restoration or its compatibility with underlying materials. This functional approach ensures that the final restoration not only looks natural (according to a set of color thresholds, for example) but also performs reliably in the oral environment.

In Step 808, engine 700 determines a rotation profile based on the data analysis conducted in Step 804. As discussed above, a rotation profile specifies the optimal orientation of the restoration within the patient's mouth, ensuring that its optical and structural properties align with the surrounding dentition. In some embodiments, Step 808's operation can involve texture mapping operations that project the analyzed optical properties onto a 3D model of the restoration.

As discussed above, the rotation profile plays a critical role in achieving seamless integration between the replacement tooth and the natural dentition. By considering factors such as the alignment of translucency gradients and surface texture patterns, engine 700's operation ensures that the restoration appears indistinguishable from the neighboring teeth. In some embodiments, Step 808 can further involve optimizing, via further analysis by the AI/ML, the orientation of the restoration to maximize its structural stability and functional performance.

In Step 810, engine 700 compiles a coloration mapping that integrates the color and rotation profiles. In some embodiments, the generated mapping can include detailed specifications for stain and glaze application, translucency gradients, and/or other optical effects. For example, analyzing the coloration mapping can further include information that is derived, determined or otherwise identified from (e.g., AI/ML analysis, for example) analysis of stain and glaze information, for which a translucency for the replacement tooth can be identified. Accordingly, the specifications of such mappings can be defined such that the desired optical properties are achieved through precise layering and material combinations of color (and/or stain and/or glaze), which as discussed above, can be compiled and/or determined via AI/ML analysis, as discussed supra, of the profiles from Steps 806 and 808.

According to some embodiments, the coloration mapping processing of Step 810 involves the generation of detailed instructions for multi-layer printing. Such instructions specify parameters such as, for example, layer thickness, material ratios, deposition patterns, and the like. By accounting for material-specific properties like viscosity and curing behavior, engine 700 can ensure that the coloration mapping is both accurate and practical. Thus, the coloration mapping provides a comprehensive blueprint for the production process, such that components of system 10 can function to provide the desired output on the replacement tooth.

In some embodiments, Step 810's mapping compilation/generation can further involve executing a predictive model that can simulate a renderable data structure (or file) that digitally represents a final appearance of the restoration under real-world conditions (as to current oral environment of the patient). Indeed, while one of ordinary skill in the art would recognize that the mapping discussed herein can be 3D mapping, as discussed supra, it should not be construed as limiting, as such mapping can be an n-based mapping, which can include, for example, a 2D mapping (as discussed supra) or a four-dimensional (4D) mapping over time, without departing from the scope of the instant disclosure.

In Step 812, engine 700 causes the execution of printer operations based on computer-executable instructions derived from the coloration mapping. For example, instructions in line with the coloration mapping in Step 810 can be provided to system 10, as discussed above respective to FIGS. 1-5. Thus, the disclosed material deposition planning translates the coloration mapping into specific commands for the printing equipment. These commands control parameters such as layer composition, thickness, and sequencing, ensuring that the desired optical properties are faithfully reproduced. Accordingly, the execution of printer operations creates restorations that closely mimic the natural appearance of teeth.

According to some embodiments, by way of non-limiting example, in accordance with the discussion above respective to at least FIG. 8, the framework can begin by conducting a thorough visual and instrumental analysis of a patient's dentition (e.g., tooth, adjacent/neighboring teeth, mouth, and the like) to identify key visual properties essential for accurate replication. This process can incorporate advanced imaging techniques, such as digital photography, intraoral scanning, and spectrophotometry, to capture high-resolution details of the tooth structure. The framework can utilize digital shade-matching tools to analyze the color distribution, translucency, and surface texture, ensuring a comprehensive assessment of the optical characteristics.

Once the initial visual properties have been identified, the framework can apply AI/ML modeling software to generate a three-dimensional representation of the patient's tooth, as discussed supra. Such software can integrate data from the imaging tools to construct an accurate model that highlights variations in hue, chroma, and value. In some embodiments, AI/ML algorithms can assist in recognizing subtle patterns in enamel and dentin, allowing for precise differentiation of shading zones and surface irregularities. The framework can further include an analysis of fluorescence and opalescence characteristics, ensuring that the synthetic replication achieves a natural aesthetic appearance.

Following such digital analysis, the framework can employ a systematic approach to selecting and applying stains and glazes to the restoration material. In some embodiments, the system can provide recommendations for specific staining techniques based on the acquired data, optimizing the layering approach to achieve the desired color depth and light reflection. The framework can incorporate a controlled application process in which stains are applied in incremental layers to replicate intrinsic and extrinsic coloration. For example, a first phase may include applying intrinsic stains to mimic deep colorations found within natural dentin, while subsequent layers can focus on enhancing enamel translucency and surface luster, and the like.

In some embodiments, the framework can integrate automated airbrushing and/or digital deposition techniques to ensure consistency in stain application. Such automated methods can precisely control stain concentration, gradient transitions, and drying times, allowing for enhanced customization and reproducibility. The framework can also employ curing protocols utilizing light or heat to stabilize the stain application before proceeding to the glazing stage.

In some embodiments, glaze applications within the framework can serve a dual purpose: i) enhancing the final aesthetics and ii) ensuring long-term durability. In some embodiments, the glaze can be customized to simulate the reflective properties of natural enamel. The framework can utilize a controlled firing process to achieve appropriate gloss levels, adjusting temperature and duration based on the material composition of the restoration, as discussed above. Additionally, the framework can include an iterative verification process, where the glazed restoration is compared to the patient's natural dentition under various lighting conditions to confirm accuracy.

In some embodiments, to further refine the restoration's appearance, the framework can integrate a polishing phase that fine-tunes the surface texture. In some embodiments, precision abrasives and diamond pastes can be employed to smooth microscopic irregularities while maintaining the integrity of the applied glaze. The framework can also include a final spectrophotometric analysis to verify that the restoration meets the predefined aesthetic parameters

Thus, in some embodiments, the framework can provide a structured, technology-driven approach to analyzing, staining, and glazing dental restorations, ensuring that the final outcome closely replicates the patient's natural dentition. By leveraging advanced imaging, digital modeling, automated application techniques, and systematic verification, the framework can achieve highly accurate and aesthetically pleasing results in synthetic dental restorations.

Accordingly, as discussed herein, the integration of the novel computational operations into the workflow of engine 700 represents a significant advancement in dental restoration technology. Each step of the Process 800—from data collection to the execution of printer operations—leverages advanced computational techniques to achieve highly personalized and accurate outcomes. By treating color matching as a multi-dimensional optimization problem, the framework overcomes the limitations of traditional methods and sets new standards for precision and efficiency in the field.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising:
identifying, by an application, data related to a set of teeth associated with a patient, the set of teeth including at least one existing tooth and a replacement tooth;
analyzing, by the application, data;
determining, by the application, based on the analysis, a color profile;
determining, by the application, based on the analysis, a rotation profile;
compiling, by the application, based on the color profile and rotation profile, a coloration mapping, the coloration mapping comprising a specification for layering of material combinations; and
executing, by the application, a printer operation based on the coloration mapping, the printer operation comprising printing on the replacement tooth according to the coloration mapping.

2. The method of claim 1, wherein the color profile corresponds to the replacement tooth, the color profile comprising information derived from a color mapping from the at least one existing tooth.

3. The method of claim 1, wherein the rotation profile corresponds to the replacement tooth, the rotation profile comprising an orientation of the patient's mouth.

4. The method of claim 1, further comprising the application analyzing the color profile and the rotation profile to determine the coloration mapping.

5. The method of claim 1, wherein the coloration mapping is a data structure comprising information related to at least one of stain or glaze.

6. The method of claim 5, further comprising analyzing the data structure, and based on the information related to at least one of stain or glaze, determining translucency for the replacement tooth, wherein the printer operation is further based on the determined translucency.

7. The method of claim 5, further comprising generating, based on the data structure, a simulation, the simulation providing a visible display of a final appearance of the restoration of the replacement tooth within current conditions of the patient's mouth.

8. The method of claim 1, wherein the coloration mapping comprises parameters selected from a group comprising: layer thickness, composition, material ratios, sequencing, and deposition patterns.

9. The method of claim 1, wherein the application comprises an artificial intelligence (AI) model.

10. A system comprising:
a processor configured to:
identify, by an application, data related to a set of teeth associated with a patient, the set of teeth including at least one existing tooth and a replacement tooth;
analyze, by the application, data;
determine, by the application, based on the analysis, a color profile;
determine, by the application, based on the analysis, a rotation profile;
compile, by the application, based on the color profile and rotation profile, a coloration mapping, the coloration mapping comprising a specification for layering of material combinations; and
execute, by the application, a printer operation based on the coloration mapping, the printer operation comprising printing on the replacement tooth according to the coloration mapping.

11. The system of claim 10, wherein the color profile corresponds to the replacement tooth, the color profile comprising information derived from a color mapping from the at least one existing tooth.

12. The system of claim 10, wherein the rotation profile corresponds to the replacement tooth, the rotation profile comprising an orientation of the patient's mouth.

13. The system of claim 10, wherein the processor is further configured such that the application analyzes the color profile and the rotation profile to determine the coloration mapping.

14. The system of claim 10, wherein the processor is further configured to analyze the coloration mapping, and based on information related to at least one of stain or glaze within the coloration mapping, determine translucency for the replacement tooth, wherein the printer operation is further based on the determined translucency.

15. The system of claim 10, wherein the coloration mapping comprises parameters selected from a group comprising: layer thickness, composition, material ratios, sequencing, and deposition patterns.

16. A non-transitory computer-readable storage medium tangibly encoded with computer-executable instructions that when executed by a device, perform a method comprising:
identifying, by an application, data related to a set of teeth associated with a patient, the set of teeth including at least one existing tooth and a replacement tooth;
analyzing, by the application, data;
determining, by the application, based on the analysis, a color profile;
determining, by the application, based on the analysis, a rotation profile;
compiling, by the application, based on the color profile and rotation profile, a coloration mapping, the coloration mapping comprising a specification for layering of material combinations; and
executing, by the application, a printer operation based on the coloration mapping, the printer operation comprising printing on the replacement tooth according to the coloration mapping.

17. The non-transitory computer-readable storage medium of claim 16, wherein the color profile corresponds to the replacement tooth, the color profile comprising information derived from a color mapping from the at least one existing tooth.

18. The non-transitory computer-readable storage medium of claim 16, wherein the rotation profile corresponds to the replacement tooth, the rotation profile comprising an orientation of the patient's mouth.

19. The non-transitory computer-readable storage medium of claim 16, further comprising the application analyzing the color profile and the rotation profile to determine the coloration mapping.

20. The non-transitory computer-readable storage medium of claim 16, further comprising analyzing the coloration profile, and based on information related to at least one of stain or glaze within the coloration mapping, determining translucency for the replacement tooth, wherein the printer operation is further based on the determined translucency.
